(19) United States
(12) Patent Application Publication
(10) Pub. No.: US 2021/0031034 A1
(43) Pub. Date: Feb. 4, 2021

(54) SYSTEMS AND METHODS FOR TREATING BRAIN DISEASE USING TARGETED NEUROSTIMULATION

(71) Applicants: Neuroelectrics Corporation, Cambridge, MA (US); BETH ISRAEL DEACONESS MEDICAL CENTER, INC. (BIDMC), Boston, MA (US)

(72) Inventors: Emiliano Santarnecchi, Boston, MA (US); Giulio Ruffini, Cambridge, MA (US); Ricardo Salvador, Cambridge, MA (US); Paul Pyzowski, Cambridge, MA (US); Alvaro Pascual-Leone, Boston, MA (US)

(21) Appl. No.: 16/969,051

(22) PCT Filed: Feb. 14, 2019
(86) PCT No.: PCT/US19/17977

§ 371 Date: Aug. 11, 2020

Related U.S. Application Data
(60) Provisional application No. 62/630,685, filed on Feb. 14, 2018.

(51) Int. Cl. A61N 1/36; A61N 1/04; A61N 2/00; A61N 2/02; A61N 7/00

(52) U.S. Cl. CPC A61N 1/36025; A61N 1/3603; A61N 1/0456; A61N 2007/0026; A61N 2/02; A61N 7/00; A61N 2/006

(57) ABSTRACT

The present invention relates to methods for treating, preventing, or slowing progression of brain diseases or disorders using targeted neurostimulation.

### FIELD OF THE INVENTION

The present invention relates to methods for treating, preventing, or slowing progression of brain diseases or disorders using targeted neurostimulation.

### BACKGROUND

Brain disease can be associated with pathological protein deposits, deficits in cognitive control, and/or deficits in neuronal circuitry. Unfortunately, therapeutic options and preventative measures are limited for these conditions. For example, since pharmaceutical agents are often poorly targeted to the brain or pathological regions of the brain, pharmaceutical interventions where available often require high doses for at best modest effects. Accordingly, methods are needed that reliably and safely treat, prevent, or slow progression of these conditions.

### SUMMARY OF THE INVENTION

The present invention provides systems and methods for treating and/or preventing brain diseases or disorders with targeted neurostimulation. The methods use target maps to provide non-invasive brain stimulation specifically to brain locations responsible for, suspected of being responsible for, or at risk of being responsible for a brain disease or disorder. In embodiments, the present invention provides for mimicking or stimulating native brain activity oscillations, patterns, and/or rhythms (such as gamma activity) in target regions of the brain to reduce or prevent pathological protein deposits and/or to improve deficits in cognitive control or neuronal circuitry.

In some embodiments, the present invention provides methods for reducing one or more protein deposit(s) in the brain of a subject. The methods include steps of obtaining or creating a target map, which identifies actual location(s) of the brain protein deposit(s) and/or likely location(s) for brain protein deposit(s), and providing a non-invasive brain stimulation in a duration; stimulation waveform(s); spatiotemporal pattern; stimulation intensity; number and type of electrode, transcranial magnetic stimulation (TMS) coil, or acoustic lens; and/or regimen sufficient to reduce one or more protein deposit(s). In some embodiments, at least one stimulation waveform is in the gamma band, and which may target one or more locations outside the sensory cortices.

In various embodiments, the present invention allows for the reduction in protein deposits or prevention in protein deposit formation, including in subjects with Alzheimer's disease (AD) or in subjects at risk of developing AD. The invention in various embodiments can be applied to the treatment or prevention of various diseases associated with protein deposits and/or with pathophysiological mechanisms associated with decreases in oscillatory activity in the gamma band and/or protein accumulation, such as interneuron pathology. These include Alzheimer's disease (AD), amyotrophic lateral sclerosis (ALS), autism (AUT), dentatorubral-pallidoluysian atrophy (DRPLA), familial amyloid cardiomyopathy (FAC), familial amyloid polyneuropathy (FAP), Huntington disease (HD), mild cognitive impairments (MCI), Parkinson's disease (PD), prion diseases or transmissible spongiform encephalopathies (TSEs), schizophrenia (SCZ), senile systemic amyloidosis (SSA), spinal bulbar muscular atrophy (SBMA), spinocerebellar ataxia type 1 (SCA1), spinocerebellar ataxia type 3 (SCA3), spinocerebellar ataxia type 6 (SCA6), spinocerebellar ataxia type 7 (SCA7), and traumatic brain injury (TBI).

In various embodiments, a target map is provided, which is developed in part from an image or scan of the subject's brain, for example, using one or more of CT, fMRI, fNIRS, MRI, PET, rs-fcMRI, and SPECT. The image or scan can employ an imaging tracer to identify protein deposits. For example, in some embodiments, a target map for the subject can be developed from positron emission tomography (PET) data and from magnetic resonance imaging (MRI) data collected from the subject.

Non-invasive brain stimulation (NIBS) is provided to the subject, specifically to engage the target map. NIBS includes tCS and its specific variants (including transcranial direct stimulation (tDCS), transcranial alternating stimulation (tACS), and transcranial random noise current stimulation (tRNS), or general field stimulation (gF-tCS), transcranial magnetic stimulation (TMS) and its specific implementations (including single pulse, monophasic or biphasic, repetitive, and burst TMS), and focused ultrasound (FUS). tCS includes any variant where each electrode may be configured to stimulate with its own unique, independent, and arbitrary waveform, only limited by current conservation. Other forms of NIBS other physical forces are also considered, for example, interaction using photons with other frequencies or using other physical force carriers.

In some embodiments, the invention provides NIBS by multichannel tCS, with electrode placement and stimulation parameters chosen to engage the target map using a standard head model. Here, one or more stimulation frequencies are in the range of 30 Hz and 120 Hz, commonly referred to as the gamma band. Alternatively, or additionally, the stimulation frequency produces gamma activity in the brain.

In some embodiments, the present invention provides the NIBS for treating a subject having or at risk of developing a disease involving cognitive deficits, or deficits in neuronal circuitry related to gamma oscillations. In embodiments, the cognitive deficits are associated with impaired activity in the gamma range. The impaired activity may be due to dysfunctional excitation-inhibition balance in cortical circuits. In embodiments, the disease is schizophrenia or autism.

Other aspects and embodiments will be apparent from the following detailed description and claims

### BRIEF DESCRIPTION OF THE DRAWINGS

FIG. 1A and FIG. 1B shows imaging useful for creating a target map. FIG. 1C shows a 3D surface reconstruction of amyloid load in a patient with Alzheimer's disease (AD). FIG. 1D shows a 3D surface reconstruction used as a target map for non-invasive brain stimulation (NIBS) and based on regions having relatively high levels of amyloid.

FIG. 2 shows the target map for the patient with AD in FIG. 1D and personalized stimulation parameters to activate the target map using transcranial current stimulation (tCS) as the NIBS method.

FIG. 3 shows amyloid levels in the patient with AD before and after tCS.

### DETAILED DESCRIPTION

In various aspects and embodiments, the invention relates to methods for treating and/or preventing brain diseases or disorders. The methods use target maps to provide non-invasive brain stimulation specifically to brain locations responsible for, suspected of being responsible for, or at risk of being responsible for the brain disease or disorder. In embodiments, the disease or disorder relates to pathological protein deposits, deficits in cognitive control, or deficits in neuronal circuitry related to gamma oscillations. In various embodiments, the present invention provides for mimicking or stimulating native brain activity oscillations, patterns, and/or rhythms (such as gamma activity) in target regions of the brain responsible for the subject's condition.

Normal electrophysiological activity in the human brain consists of oscillatory activity across a wide range of frequencies, with oscillatory activity in the 30-120 Hz range referred to as “gamma” activity (also herein referred to as the “gamma band”). Dysregulation of gamma activity linked to interneurons' pathology and pathologic network hyperexcitability has been observed in animal models of AD. In various embodiments, the invention delivers stimulation (using non-invasive brain stimulation, or NIBS) in the quasi-static regime to targeted locations. For example, the stimulation waveform(s) may be delivered in the gamma band, e.g., between about 30 Hz and about 120 Hz. In various embodiments, the NIBS is directed to at least one location outside of the auditory cortex and/or the visual cortex in a duration; stimulation waveform(s); spatiotemporal pattern; stimulation intensity; number and type of electrode, TMS coil, or acoustic lens; and/or regimen sufficient to reduce one or more protein deposit(s), to improve deficits in cognitive control, or to improve neuronal circuitry.

Transcranial current stimulation (tCS) is a form of non-invasive brain stimulation (NIBS) that uses electrodes placed on the scalp to deliver weak electrical currents to the brain. tCS is used to stimulate or inhibit one or more target brain region(s). tCS includes a family of related non-invasive techniques such as transcranial direct stimulation (tDCS), transcranial alternating stimulation (tACS), transcranial random noise current stimulation (tRNS), general field stimulation (gF-tCS), or any other form of multichannel current stimulation. This includes a variant where each electrode may be configured to stimulate with a unique, independent, and arbitrary waveform only limited by current conservation, with stimulation waveform band-limited to <10 kHz. For example, such a variant may include amplitude modulated waveforms as described in Witkowski et al., Neuroimage, 2016. or use of interfering fields to reach deeper targets as described in Grossman et al., Cell, 2017.

NIBS also includes transcranial magnetic stimulation (TMS) and its specific implementations (including single pulse, monophasic or biphasic, repetitive, and burst TMS), focused ultrasound (FUS), or any other form of non-invasive stimulation that can be administered to interact with and stimulate neuronal populations.

Each NIBS used in the present invention is capable of mimicking and/or stimulating native brain activity oscillations, patterns, and/or rhythms, e.g., gamma activity.

In accordance with the invention, NIBS can interact with brain oscillations by means of tCS where low current intensity (max 2 mA) alternating sinusoidal currents are applied via scalp electrodes. For example, tACS is a promising technique to modulate activity in healthy and/or pathological brains due to its inherent safety (because of its non-invasiveness and the low intensities used, which are at least an order of magnitude below the intensity which causes tissue damage) and controllability (in terms of stimulation frequency and its ability to target nearly any cortical region). In animals, tACS can entrain neurons (induce synchronized activity) in a variety of cortical areas. More general stimulation waveforms, particularly those derived from endogenous activity, can be particularly effective in engaging neuronal populations in slices. Stimulations, supported by electroencephalography (EEG) evidence, demonstrate that tACS modulates brain oscillatory activity via network resonance, suggesting that stimulation at a resonant frequency could cause large-scale modulation of network activity and could amplify endogenous network oscillations in a frequency-specific manner.

Numerous neurodegenerative or neurological diseases are known to be associated with pathological protein deposits and interneurons'-related pathological decrease in gamma oscillations; see, e.g., Kaytor and Warren et al., JBC 1999. Examples of diseases associated with protein deposits and/or interneurons' pathology and/or decrease in gamma oscillations, include Alzheimer's disease (AD), amyotrophic lateral sclerosis (ALS), autism (AUT), dentatorubral-pallidoluysian atrophy (DRPLA), familial amyloid cardiomyopathy (FAC), familial amyloid polyneuropathy (FAP), Huntington disease (HD), mild cognitive impairments (MCI), Parkinson's disease (PD), prion diseases or transmissible spongiform encephalopathies (TSEs), schizophrenia (SCZ), senile systemic amyloidosis (SSA), spinal bulbar muscular atrophy (SBMA), spinocerebellar ataxia type 1 (SCA1), spinocerebellar ataxia type 3 (SCA3), spinocerebellar ataxia type 6 (SCA6), a spinocerebellar ataxia type 7 (SCA7). In these and related diseases, the protein deposit(s) may include one or more of α-synuclein, amyloid (e.g., amyloid-β), ataxin-1, ataxin-3, ataxin-7, atrophin-1, Fused in Sarcoma/Translocated in Sarcoma (FUS/TLS), huntingtin, polyglutamine-expanded androgen receptor (polyQ-AR), prion protein (PrP), spinocerebellar ataxia type 6-associated calcium channel, superoxide dismutase 1 (SOD1), TAR DNA-binding protein 43 (TDP-43), Tau protein, transthyretin (TTR), traumatic brain injury (TBI), and ubiquinated proteins. Unfortunately, drug-based interventions for these diseases, if available, do not allow precise targeting of the protein deposits; therefore, higher doses and/or more frequent dosing may be required to achieve a therapeutic effect; further, these effects are rather modest. Moreover, the drug-based interventions are not usually begun until a subject has been diagnosed with the disease; thus, the drugs are not normally used as preventative measures. Indeed, there exist few, if any, prophylactics for diseases associated with protein deposits.

A particularly devastating neurodegenerative disease is Alzheimer's disease (AD). Pharmacologic interventions for AD only transiently improve function; currently, there are no available treatments or preventative measures that alter or cease AD progression. AD is associated with a cascade of effects including initial interneurons'-related pathology, leading to a decrease in gamma oscillations and pathological protein deposits, i.e., deposits of amyloid protein (e.g., amyloid plaques) and Tau protein deposits (as neurofibrillary tangles). Recent animal work indicates that visual stimulation (in the gamma band, i.e., ˜40 Hz) may modulate interneurons' activity, leading to increase in gamma activity and reduce accumulation of amyloid protein deposits and Tau levels in the visual cortex of mouse models of AD (Iaccarino et al., Nature 2016). However, such sensory stimulation is limited to protein deposits located in sensory cortices.

There exists an unmet need for methods to treat and/or prevent diseases associated with accumulation of protein deposits in the brain, including regions outside the sensory cortices.

In certain aspects of the disclosure, the methods involve obtaining or developing a map comprising locations in the brain where protein deposits generally are known/believed to occur and/or commonly occur in patients having the disease or specifically occurring in a particular patient, and then using a NIBS targeted to the locations to reduce the amount of protein deposits. Although proteins involved and disease progression may differ, a focus on reducing, preventing, or elimination such protein deposits may be an avenue for clinical treatments of or preventative measures for the diseases described herein.

In various embodiments, the target map identifies actual location(s) of the brain protein deposit(s) and/or likely location(s) for brain protein deposit(s). In these embodiments, NIBS can be provided in a duration; stimulation waveform(s); spatiotemporal pattern; stimulation intensity; number and type of electrode, TMS coil, or acoustic lens; and/or regimen sufficient to reduce one or more protein deposit(s).

Aspects of the present invention relate to methods for treating a subject having a disease associated with protein deposits, and/or interneurons' pathology, and/or decreases in gamma oscillations, reducing a symptom of the disease, and/or preventing progression of the disease. Examples of such diseases associated with protein deposits include Alzheimer's disease (AD), amyotrophic lateral sclerosis (ALS), autism (AUT), dentatorubral-pallidoluysian atrophy (DRPLA), familial amyloid cardiomyopathy (FAC), familial amyloid polyneuropathy (FAP), Huntington disease (HD), mild cognitive impairments (MCI), Parkinson's disease (PD), prion diseases or transmissible spongiform encephalopathies (TSEs), schizophrenia (SCZ), senile systemic amyloidosis (SSA), spinal bulbar muscular atrophy (SBMA), spinocerebellar ataxia type 1 (SCA1), spinocerebellar ataxia type 3 (SCA3), spinocerebellar ataxia type 6 (SCA6), spinocerebellar ataxia type 7 (SCA7), and traumatic brain injury (TBI).

The subject having a disease associated with protein deposits may be determined using a behavioral, cognitive, and/or physiological test. The subject having the disease may be symptomatic or asymptomatic, for example, a subject with Parkinson's disease may exhibit a hand tremor or s/he may not yet exhibit a tremor. The subject having the disease may have detectable protein deposits in his/her brain and has clinical or preclinical disease. Alternatively, the subject having the disease may not have detectable protein deposits in his/her brain.

Recent positron emission tomography (PET) imaging studies of AD patients suggest progressive amyloid deposition can begin up to twenty years before the onset of clinical symptoms, with deposition stabilizing around the time that clinical symptoms begin to be prominent. Using AD as a non-limiting example, treatments (as described herein) may be given prophylactically and when a subject is asymptomatic for AD and/or before or at the start of amyloid deposition; such prophylactic treatments may be useful in preventing development and/or progression of AD.

Accordingly, aspects of the present invention also relate to methods for preventing a subject from acquiring and/or developing a disease associated with protein deposits, as described herein. This subject may be referred to as a subject at risk for developing a disease associated with protein deposits. The subject at risk for developing a disease associated with protein deposits may be determined using a behavioral, cognitive, and/or physiological test. Typically, the subject is asymptomatic for the disease but may be symptomatic for markers/indicators that are predictive of the disease or markers/indicators which classify the subject as being in a pre-diseased state. The subject at risk for the disease may have detectable protein deposits in his/her brain, yet, behavioral, cognitive, and/or physiological tests may not identify the subject as having the disease; alternately, the subject at risk may not have detectable protein deposits in his/her brain. Additionally, a subject may be considered “at risk” due to his/her age, diet, health status, other medical diseases/disorders, family history, and/or genetic characteristics/genetic profile. Subjects at risk for developing the disease associated with protein deposits may be provided the treatment methods, as described herein, yet with a prophylactic focus. These prophylactic and preventative measures ensure that the subject at risk slows and/or ceases protein depositing which could lead to or develop into a neurological disorder, as described herein.

In diseases associated with protein deposits, the protein deposited may include one or more of α-synuclein, amyloid (e.g., amyloid-β), ataxin-1, ataxin-3, ataxin-7, atrophin-1, Fused in Sarcoma/Translocated in Sarcoma (FUS/TLS), huntingtin, polyglutamine-expanded androgen receptor (polyQ-AR), prion protein (PrP), spinocerebellar ataxia type 6-associated calcium channel, superoxide dismutase 1 (SOD1), TAR DNA-binding protein 43 (TDP-43), Tau protein, transthyretin (TTR), and ubiquinated proteins.

As discussed above, Alzheimer's disease (AD) is a disease associated with protein deposits, with the protein deposits comprising amyloid protein and/or Tau protein. Evidence suggests that both amyloid and Tau play a role in AD pathogenesis. Thus, interventions that reliably and safely decrease the intracerebral burden of amyloid and/or Tau could be of clinical importance.

An aspect of the present invention is a method for reducing amyloid and/or Tau protein deposits in the brain of a subject. The method includes steps of obtaining a target map comprising actual location(s) of amyloid and/or Tau protein deposits in the subject's brain and densities thereof and/or likely location(s) of amyloid and/or Tau protein deposits in the subject's brain and providing a NIBS to the actual location(s) and/or likely location(s) in a duration; stimulation waveform(s); spatiotemporal pattern; stimulation intensity; number and type of electrode, TMS coil, or acoustic lens; and/or regimen sufficient to reduce the amyloid and/or Tau protein deposits.

In embodiments, methods result in one or more of improving memory, cognition, behavior, and/or motor functions in a subject having a disease associated with protein deposits (including an asymptomatic subject), in a pre-symptomatic subject, and/or in a subject at risk for developing a disease associated with protein deposits.

In embodiments, the NIBS, as described herein, increases microglia activation which results in clearance of protein deposits.

Aspects of the present invention relate to methods for treating a subject having a disease involving cognitive deficits. The cognitive defects may be associated with impaired activity in the gamma range. The impaired activity may be due to dysfunctional excitation-inhibition balance in cortical circuits. The methods further include reducing a symptom of the disease and/or preventing progression of the disease. Aspects of the present invention also relate to methods for treating a subject having a disorder associated with deficits in neuronal circuitry related to gamma oscillations, reducing a symptom of the disorder, and/or preventing progression of the disorder.

Without wishing to be bound by theory, diseases and symptoms thereof associated with deficits in cognitive control (e.g., schizophrenia) can stem from impaired prefrontal gamma oscillations. Recent data in humans (Woo et al., Harv Rev Psychiatry, 2010; Cho et al., PNAS, 2006; and Lewis Eur J Neurosci., 2012), suggest an intrinsic deficit (of genetic origin) in pyramidal neuron dendritic spines (Glausier and Lewis, Neuroscience, 2013). The associated loss of excitatory synapses and the resulting reduction in cortical network activity leads to a homeostatic reduction in inhibition from parvalbulmin basket cells (PV) in Layer 3 to help restore excitatory-inhibitory balance. Together, these result in pathophysiologically-diminished gamma oscillations and clinical syndromes characterized by deficient cognition.

Certain disorders associated with impaired cortical circuitry may be due to excitation-inhibition deficits affecting fast rhythms. One example of such a disorder is autism (Rojas and Wilson, Biomark Med., 2014 and Hashemi et al., Cereb. Cortex, 2017), in which the number of parvalbumin-expressing interneurons is decreased in the medial prefrontal cortex.

Accordingly, aspects of the present invention relate to NIBS treatments (e.g., tCS) to drive and entrain oscillations in neurons lacking proper prefrontal gamma oscillations or deficits in neurons/circuits related to such gamma oscillations. Here, the stimulation waveforms recover some functioning of the underlying circuits and produce plastic changes that enable partial recovery of the diseases or disorders, including improvements in cognition and/or behavior.

In embodiments of the present invention, where exogenous electric fields applied via NIBS are dynamic, the mechanism for long term effects may be associated with spike-timing-dependent plasticity (STDP). Thus, NIBS treatment may be particularly helpful in early phases of a disease, since gamma deficits may affect synaptic reorganization development, especially during the period of late adolescence and early adulthood (Woo et al., 2010). For this reason, prophylactic treatments for such types of disease are particularly appropriate for certain classes of patients (e.g., those with genetic predisposition for diseases associated with deficits in cognitive control or disorders associated with impaired cortical circuitry) and possibly in synergistic combination with gene therapy and/or in combination with drug interventions.

Consequently, aspects of the present invention also relate to methods for preventing a subject from acquiring and/or developing a disease involving cognitive deficits associated with impaired activity in the gamma range and/or for preventing a disorder associated with deficits in neuronal circuitry related to gamma oscillations. This subject may be referred to as a subject at risk for developing a disease/disorder. The subject at risk for developing a disease or disorder may be determined using a behavioral, cognitive, and/or physiological test. Typically, the subject is asymptomatic for the disease/disorder but may be symptomatic for markers or indicators that are predictive of the disease/disorder or markers or indicators which classify the subject as being in a pre-diseased/disordered state, even when the behavioral, cognitive, and/or physiological tests have not identified the subject as having the disease/disorder. Additionally, a subject may be considered “at risk” due to his/her age, diet, health status, other medical diseases/disorders, family history, and/or genetic characteristics/genetic profile. Subjects at risk for developing the disease/disorder may be provided the treatment methods, as described herein, yet with a prophylactic focus. These prophylactic and preventative measures ensure that the subject at risk slows and/or ceases progress of the disease/disorder which could lead to or develop into a neural deficit, as described herein.

In aspects or embodiments, a target map may include a catalog of locations in the brain that may be targeted with a non-invasive brain stimulation, a catalog of the temporal characteristics of the electric field at the location, and a weight map assigned to each location. In embodiments, the target map is the catalog of two fields: the targeted electric field E(x,t) and the weight map W(x,t), where bold indicates a vector.

In embodiments, the target map defines a desired spatiotemporal stimulation pattern for the subject. The number and type (i.e., montages) of electrodes, TMS coils, or acoustic lenses, may be used to deliver a spatiotemporal stimulation pattern, optionally using a genetic algorithm. Genetic algorithms are described, for example, in U.S. Pat. No. 9,694,178, which is hereby incorporated by reference in its entirety.

In embodiments, an electrode, TMS coil, or acoustic lens montage comprises, respectively, a specified number of electrodes TMS coils, and acoustic lenses, specified location of electrodes, TMS coils, and acoustic lenses, as well as specified stimulation parameters (as described herein). Determination of number and location of electrodes and optimal stimulation parameters to stimulate multiple targets at once is described in US 2015/0112403 (now U.S. Pat. No. 9,694,178), the entire disclosure of which is hereby incorporated by reference.

Using electrode montage as an example of the three types of montages described herein, generally, the optimization of stimulation parameters, electrode locations and electrode numbers can employ extended, weighted cortical pattern target maps based on brain activity data and/or neuroimaging data. The target maps define desired values for the electric field at multiple spatial and temporal points for stimulation. Targets can be defined based on a coordinate system relative to the cortical surface, with target values for normal and/or tangential components of electric field to the cortex, or, more generally, by a spatiotemporal field in the brain. The process can use algorithms to optimize currents, for example, as well as the number and location of electrodes given appropriate constraints, such as the maximum current at any electrode and the maximum total injected current. For example, an electrode montage and stimulation parameters to be provided can be determined using a target map of a cortical surface specifying desired values for the electric field at each (spacetime) point. Further, determination of an electrode montage and stimulation parameters to be provided can employ a weight map providing the degree of relative importance of each location in the target map, and a set of constraints on the number of electrodes and their currents. In embodiments, the weighted target map of the cortical surface is generated by prioritizing the areas in the target map for optimization purposes. For example, a higher weight is given to those brain areas considered to be more important for the particular application of neurostimulation.

In embodiments, the calculation of stimulation parameters and electrode locations is performed under constraints regarding maximal electrode number, maximal or minimal current at each electrode, and the total current injected into the brain by all electrodes at any time. In embodiments, the calculations are performed under additional constraints including holding the current in an electrode at a constant fixed value.

In embodiments, the calculation of stimulation parameters (e.g., current intensity for tCS) uses least squares. In embodiments, the present method comprises using constrained least squares to optimize current intensities, as an example. Exemplary methods for current optimization are described in US 2015/0112404 (now U.S. Pat. No. 9,694,178), the entire disclosure of which is hereby incorporated by reference.

In embodiments, the calculation of optimal electrode locations and/or optimal electrode numbers employs a genetic algorithm. Genetic algorithms are described, for example, in US 2015/0112403 (now U.S. Pat. No. 9,694,178), the entire disclosure of which is hereby incorporated by reference. The genetic algorithm can be based on the definition of a solution by a “DNA” binary string (in this case of dimension N-1) specifying the electrode locations and number, and stimulation parameters, and may employ as an optimization function the least squares error, i.e., the one with the best possible current configuration for the chosen electrode locations. Cross-over and mutation functions are defined to ensure that the offspring of solutions do not violate the constraint of maximal number of electrodes in the solution. Once a DNA string is specified (i.e., a particular montage), its fitness can be computed by inverting the solution for that particular montage. Solutions with more than the maximal number of electrodes desired are penalized strongly. The genetic algorithms with specifically designed fitness, cross-over and mutation functions, converge quickly and reliably to a solution.

In embodiments, the target map is based upon a brain image or scan of the subject. The image or scan may be CT, EEG, ERPs, MRI, fNIRS, MEG, MRI, PET, rs-fcMRI, SPECT, theta-burst rTMS, TMS/EEG, or TMS/MEPs, or a combination thereof. Such a personalized brain image or scan allows for single patient characterization and without a need for use of reference databases. Thus, NIBS may be provided in locations and under parameters (as described herein) that are optimized to the patient.

In embodiments, the image or scan includes use of an imaging tracer, e.g., which identifies protein deposits. In a non-limiting example, the imaging tracer identifies amyloid and/or Tau protein deposits, e.g., for AD-related methods. Examples of imaging tracers include Florbetaben (Neuraceq®), Florbetapir (Amyvid®), Flutemetamol (Vizamyl®), Pittsburgh compound B (PIB), e.g., [¹¹C]PiB, and [¹⁸F]T807. In embodiments, the imaging tracer is [¹¹C]PBR28.

Other tracers suitable for imaging one or more of the deposited proteins described herein may be used. Image tracers are especially suited for use with PET. In these embodiments, the target map identifies the precise locations of protein deposits; thus, the NIBS may be targeted primarily to those locations. Additionally, the severity of the protein deposits may be determined and used to vary stimulation parameters, e.g., duration; stimulation waveform(s); spatiotemporal pattern; stimulation intensity; number and type of electrode, TMS coil, or acoustic lens; regimen; and/or NIBS type, e.g., tCS type.

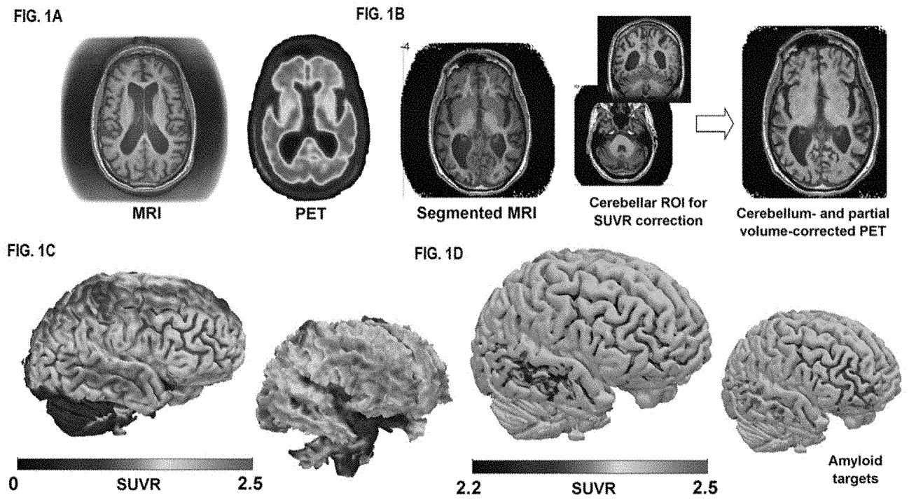

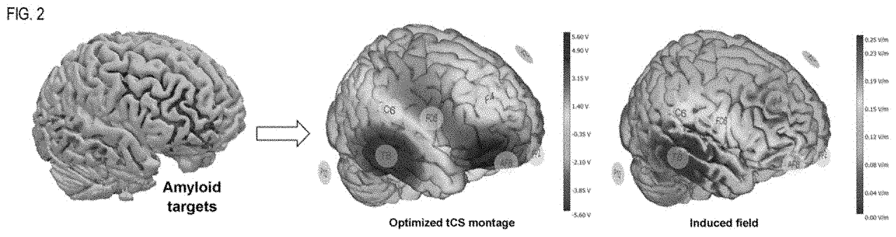

Embodiments of the present invention may include PET with partial volume correction, based on cerebral and cerebellar individual grey/white matter masks, which helps produce more accurate maps and which show protein deposit variations at the sulcal/gyral level. PET may help provide for stimulation intensity correction, e.g., correction for cerebellar grey matter using spatial clustering. Such targeted and tailored approaches improve efficiency and effectiveness of the present methods. See, FIG. 1 and FIG. 2.

Such protein deposition data may be converted into a target map for optimized NIBS, i.e., via a standard head model or to a personal realistic head model for the patient, e.g., from an MRI of the patent. See, e.g., Miranda et al., 2013.

In embodiments, an optimization procedure is used to target NIBS generated fields on the cortex of a subject's brain. Computational models of brain function and dysfunction may play a key role in reducing risk and uncertainty in clinical trials and provide the means for personalized therapies that account for individual biophysical and physiological characteristics. This can be achieved by incorporating a mechanistic understanding of the effects of NIBS (and, in embodiments, along with drugs) within realistic brain models, thereby enabling the effective development of synergistic, individualized therapies. Aspects of the optimization procedures have been described in WO2015/059545. Also described therein are uses of optimized multichannel tCS that preferentially engage the target map; this target map includes the locations that propagate activation signals upon stimulation, locations that propagate inhibition signals upon stimulation, and neutral locations, which may be avoided.

In embodiments, optimal currents, optimal electrode locations, and/or optimal electrode numbers is determined using a realistic head model with electric field modeling. In embodiments, the electric field calculations are performed using the realistic head model described in Miranda et al., (2013). In embodiments, the realistic head model is a multilayer finite element model of a realistic head that may be either generic or specific to a patient, e.g., from an MRI of the patient. In such embodiments, tissue boundaries are derived from MR images (e.g., scalp, skull, cerebrospinal fluid (CSF) including ventricles, Grey Matter, and White Matter) with or without CT scans, and the finite element method is used to calculate the electric potential in the head, subject to the appropriate boundary conditions. Tissues are assumed to be uniform and isotropic, and values for their electric conductivity may be obtained from the literature.

In embodiments, the target map is defined for the cortical (Grey Matter-CSF or White Matter-Grey Matter interface surface) normal component of the electric field or for the electric field's absolute value. Generally, the target map could be defined for any function of the electric field. Relative phase of the electric field in different positions may also be considered when preparing a target map and/or when optimizing stimulation parameters.

In embodiments, electroencephalography (EEG) and/or magnetoencephalography (MEG) are further used to determine the stimulation parameters (as described herein).

In embodiments, a target map is better defined using neuro-computational models of the brain.

Beyond a biophysical model of the brain, a physiological computational model of the subject's brain (see, e.g., Merlet et al., PLOS One 2013) derived from electrophysiological and biophysical data may also be used to define the target map to optimize the montage specifications and currents (as generally described in WO2015/059545), including intensities and frequencies, or more generally, personalized stimulation spatiotemporal electric field patterns.

In embodiments, the target map is based upon brain images or scans from a cohort of subjects. The image or scan may be CT, EEG, ERPs, fMRI, fNIRS, MEG, MRI, PET, rs-fcMRI, SPECT, theta-burst rTMS, TMS/EEG, or TMS/MEPs, or a combination thereof.

In embodiments, the target map is based upon where protein deposits generally are known/believed to occur and/or commonly occur in patients having the subject's disease/disorder.

Recent animal work indicates that visual stimulation in the gamma band may alleviate the accumulation of amyloid protein deposits in the visual cortex. Exogenously-induced 40 Hz gamma oscillations reduce Aβ levels and amyloid plaques, and may also reduce Tau levels, in the visual cortex of mouse models of AD. Moreover, in pre-symptomatic AD mice, induction of gamma activity may prevent subsequent neurodegeneration and behavioral deficits. Further, in AD patients, changes in brain connectivity in the gamma band (as measured with EEG) are observed after administration of antiepileptic drugs.

Normal electrophysiological activity in the human brain consists of oscillatory activity across a wide range of frequencies, with oscillatory activity in the 30-120 Hz range referred to as “gamma” activity (also herein referred to as the “gamma band”). Patients with AD often have relative attenuation of gamma frequency activity. Dysregulation of gamma activity linked to interneurons' pathology and pathologic network hyperexcitability has been observed in animal models of AD.

In healthy humans, gamma band (e.g., about 40 Hz) stimulation of the prefrontal cortex induces behavioral effects, including an increase in abstract reasoning abilities, which is a cognitive function demonstrated to be linked to fast gamma oscillatory activity. This effect has been shown to be stimulation-frequency specific and evidence suggests that the effect is due to entrainment of spontaneous gamma oscillations in the brain. tACS at 60 Hz and 80 Hz (i.e., “high-gamma”) stimulating the motor cortex modulates visuo-motor performance in healthy participants. Additional evidence also suggests the possibility of increasing gamma oscillations in the temporal lobe, with significant long-lasting modifications of ongoing gamma spectral power after stimulation.

In embodiments, the NIBS delivers a stimulation in the quasi-static regime (less than about 10,000 Hz) to the location. In embodiments, the stimulation waveform(s) delivered is in the gamma band, e.g., between about 30 Hz and about 120 Hz. In embodiments, the stimulation is between about 40 Hz and about 50 Hz.

In embodiments, the stimulation includes more than one distinct stimulation waveform, e.g., including at least one frequency in the gamma band or at least one frequency in the gamma band and at least one frequency outside the gamma band. In embodiments, each frequency is in the gamma band. In embodiments, at least one frequency is a non-sinusoidal waveform, e.g., the non-sinusoidal waveform is in the gamma band. In embodiments, the stimulation includes random and/or varying frequencies.

In embodiments, the frequency is outside the gamma band, e.g., in the theta band or delta band, yet the stimulation waveform produces gamma activity at or near the location of the stimulation.

In embodiments, the stimulation has a duration of at least 1 second, at least 1 minute, or at least 1 or 2 hours, e.g., from about 5 minutes to about 1 hour.

In embodiments, the regimen (of NIBS) includes only one session. Alternatively, the regimen includes more than one session, with the sessions being annual, bimonthly, monthly, semimonthly, biweekly, weekly, semiweekly, daily, or more than daily

In embodiments, the non-invasive brain stimulation (NIBS) is provided via transcranial current stimulation (tCS), transcranial magnetic stimulation (TMS), or transcranial focused ultrasound (FUS). In embodiments, the tCS is selected from transcranial direct current stimulation (tDCS), transcranial alternating current stimulation (tACS), and random noise current stimulation (tRNS), or general field stimulation (gF-tCS). In embodiments, the tCS is tACS, e.g., the tACS is a multichannel tACS. In embodiments, at least two channels in the multichannel tACS have different stimulation parameters (including stimulation waveform and/or intensities). In embodiments, each channel has the same stimulation parameter.

In embodiments, the methods of the invention further include providing an error tolerance map on the brain's cortex.

In embodiments, the stimulation parameters, e.g., in a duration; stimulation waveform(s); spatiotemporal pattern; stimulation intensity; number and type of electrode, TMS coil, or acoustic lens; regimen, and/or NIBS type, for the NIBS may be varied based upon the disease/disorder itself, the progress of the disease/disorder, and health/disease state characteristics (e.g., age, family history, and presence/absence of symptoms) of the subject; for diseases/disorders associated with protein deposits, the location of protein deposits (if any) and/or the severity of the protein deposits.

In embodiments, the stimulation is directed to at least one location outside of the auditory cortex and/or the visual cortex.

In embodiments, the stimulation's electric field target is the perpendicular or normal electric field to the surface on the cortex of the brain of the subject.

In embodiments, the stimulation entrains gamma activity in the brain of the subject.

In embodiments, the stimulation is at another frequency (e.g., delta or theta) which enhances gamma activity. For example, the other frequency is theta and it leverages theta-gamma phase-amplitude coupling.

In embodiments, gamma activity resulting from the NIBS stimulation occurs at or near the location of the stimulation, e.g., no more than 10 cm, no more 1 cm, no more 1 mm, no more 100 μm, or no more 10 μm from the stimulation.

In embodiments, the subject is undergoing or has undergone a pharmaceutical or non-pharmaceutical therapy for the disease associated with protein deposits, e.g., AD. In embodiments, the subject is recommended, provided, and/or administered a pharmaceutical or non-pharmaceutical therapy for the disease associated with protein deposits, e.g., AD.

In embodiments, the stimulation, e.g., tCS, has a current intensity between about 0.1 mA and about 10 mA or between about 0.01 A/n to about 100 A/m².

In embodiments, the NIBS, e.g., tCS, is provided via at least one electrode, e.g., via an electrode montage comprising more than one electrodes. In embodiments, at least two electrodes in the electrode montage have different stimulation parameter (including stimulation waveform and/or intensities). In embodiments, each electrode in the electrode montage has the same stimulation parameters. The electrode montage may include up to 2, up to 4, up to 8, up to 16, up to 32, up to 64, up to 128, or up to 256 electrodes. The electrode montage may include from about 1 to about 300, about 1 to about 100, about 1 to about 50, about 1 to about 40, about 1 to about 30, about 1 to about 25, about 1 to about 20, from about 1 to about 15, from about 1 to about 10, from about 2 to about 8, or from about 4 to about 8 electrodes.

In embodiments, the present methods include calculating a minimal number of electrodes needed for providing tCS based on the target map and/or a target map and an error tolerance map.

Any aspect or embodiment described herein can be combined with any other aspect or embodiment as disclosed herein.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example, within plus or minus 10%.

The invention is further described in accordance with the following non-limiting example.

### EXAMPLE

### Example: Administering a Non-Invasive Brain Stimulation to a Human Subject with Alzheimer's Disease Reduces Pathological Protein Deposits

In this example, a human subject with Alzheimer's disease (AD) was treated with a non-invasive brain stimulation (NIBS) according to the present invention. The NIBS treatment reduced amyloid deposits in the subject's brain.

A protein target map for the human subject was developed by identifying amyloid protein targets based on PET and MRI imaging data. FIG. 1A shows structural MRI and amyloid PET images. Here, the MRI is segmented in grey and white matter tissue classes for partial-volume correction of PET data, and the average tracer uptake from the cerebellar gray matter is used as reference value, leading to a cerebellum-corrected PET image shown in FIG. 1B. A 3D surface reconstruction of the amyloid load in the human subject is shown in FIG. 1C. Finally, a threshold for cortical amyloid Relative Standard Uptake Value (SUVR) was set to identify regions with maximal level of amyloid to be used as a target map for the non-invasive brain stimulation by the tCS method. See, FIG. 1D.

Shown in FIG. 2 is the personalized NIBS stimulation parameters, using eight stimulating electrodes, to activate the target map of the human subject. The location of the electrodes and the stimulation waveform were optimized using a realistic electrical head model; in this example the model was derived from a finite element model based on the MRI of the human subject. Here, the targets were selected to optimize a multi-electrode transcranial current stimulation (tCS) montage aimed at maximizing the electric field in the target regions, while minimizing it over the rest of the brain.

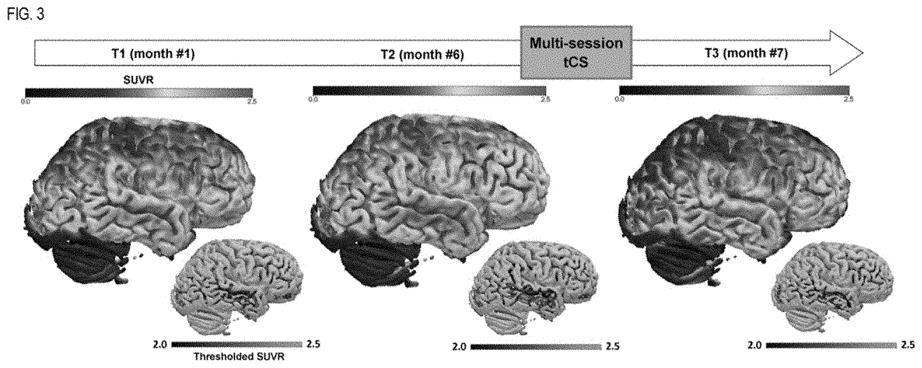

FIG. 3 shows a drastic reduction in amyloid deposits in the human subject's brain following NIBS (i.e., a multi-session tCS) according to the present invention. FIG. 3 shows three representations of the human subject's brain and amyloid depositing therein, taken at month one (T1), month six (T2), and month seven (T3; after treatment with tCS), in each pair of representations, the larger picture shows the results of amyloid uptake via a tracer, whereas the smaller picture shows the same data on a color scale selected to highlight the regions of pathologic amyloid. The T1 representation shows the combined PET/MRI data at baseline. The T2 representation shows a substantial increase in amyloid protein deposits, which is consistent with the known progression of AD (VilleMagne et al., Lancet Neurology, 2013). After the images were taken for T2, the human subject was administered ten daily sessions of NIBS (here tCS), each session lasting approximately 30 minutes. The T3 representation, taken one month after start of the tCS treatments, shows a significant reduction in amyloid deposits compared to T2 and also when compared to T1 (i.e., the baseline time point).

In summary, administering NIBS according to the present invention to the human subject with Alzheimer's disease reduced pathological protein deposits in the subject's brain to levels below baseline.

### Equivalents

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### Incorporation by Reference

All patents and publications referenced herein are hereby incorporated by reference in their entireties. Exemplary publications are listed below in the “REFERENCES” section and throughout the above-disclosure.

The publications discussed herein are provided solely for their disclosure prior to the filing date of the present application. Nothing herein is to be construed as an admission that the present invention is not entitled to antedate such publication by virtue of prior invention.

### REFERENCES

Fröhlich, McCormick “Endogenous electric fields may guide neocortical network activity.” Neuron. 67(1):129-43, 2010
Grossman, et al., “Noninvasive Deep Brain Stimulation via Temporally Interfering Electric Fields”, Cell, 169(6):1029-1041, 2017
Iaccarino, Singer, Martorell, Rudenko, Gao, Gillingham, Hansruedi mathys, Jinsoo Seo, Oleg Kritskiy, Fatema Abdurrob, Chinnakkaruppan Adaikkan, Rebecca G. Canter, Richard Rueda, Emery N. Brown, Edward S. Boyden & Li-Huei, “Gamma frequency entrainment attenuates amyloid load and modifies microglia.” NATURE, VOL 540, 2016
Merlet, Birot, Salvador, Molaee-Ardekani, Mekonnen, Soria-Frish, Ruflini, Miranda, Wendling, “From oscillatory transcranial current stimulation to scalp EEG changes: a biophysical and physiological modeling study.” PLoS ONE January 2013; 8(2):e57330
Miranda, Mekonnen, Salvador, Rufini, “The electric field in the cortex during transcranial current stimulation.” NeuroImage 12/2012
Ruffini, Fox, Ripolles, Miranda, Pascual-Leone, “Optimization of multifocal transcranial current stimulation for weighted cortical pattern targeting from realistic modeling of electric fields.” Neuroimage 2014
Ruffini, Wendling, Merlet, Molaee-Ardekani, Mekkonen, Salvador, Soria-Frisch, Grau, Dunne, Miranda, “Transcranial Current Brain Stimulation (tCS): Models and Technologies.” IEEE transactions on neural systems and rehabilitation engineering: a publication of the IEEE Engineering in Medicine and Biology Society. September 2012
Santarnecchi, Biasella, Tatti, Rossi, Prattichizzo, and Rossi, “High-gamma oscillations in the motor cortex during visuo-motor coordination: A tACS interferential study.,” Brain Research Bulletin. vol. 131, pp. 47-54, 2017
Santarnecchi, N. R. Polizzotto, M. Godone, et al., “Frequency-Dependent Enhancement of Fluid Intelligence Induced by Transcranial Oscillatory Potentials.,” Current Biology. vol. 23, no. 15, pp. 1449-1453, 2013
Santarnecchi, T. Muller, S. Rossi, et al., “Individual differences and specificity of prefrontal gamma frequency-tACS on fluid intelligence capabilities.,” Cortex. vol. 75, pp. 33-43, 2016
Witkowski et al., “Mapping entrained brain oscillations during transcranial alternating current stimulation (tACS).” Neuroimage. 15; 140: 89-98, 2015

What is claimed is:

1. A method for reducing one or more protein deposit(s) in the brain of a subject comprising:
obtaining a target map, wherein the target map identifies actual location(s) of the brain protein deposit(s) and/or likely location(s) for brain protein deposit(s); and
providing a non-invasive brain stimulation (NIBS) in a duration; stimulation waveform(s); spatiotemporal pattern; stimulation intensity; number and type of electrode, TMS coil, or acoustic lens; and/or regimen sufficient to reduce one or more protein deposit(s).

2. The method of claim 1, wherein the protein deposit(s) comprises one or more of α-synuclein, amyloid (e.g., amyloid-β), ataxin-1, ataxin-3, ataxin-7, atrophin-1, Fused in Sarcoma/Translocated in Sarcoma (FUS/TLS), huntingtin, polyglutamine-expanded androgen receptor (polyQ-AR), prion protein (PrP), spinocerebellar ataxia type 6-associated calcium channel, superoxide dismutase 1 (SOD1), TAR DNA-binding protein 43 (TDP-43), Tau protein, transthyretin (TTR), and a ubiquinated protein.

3. The method of claim 2, wherein the protein deposit(s) comprises amyloid and/or Tau protein.

4. The method of claim 3, wherein the protein deposit(s) comprises amyloid-β.

5. The method of claim 4, wherein the protein deposit(s) comprises phosphorylated Tau protein.

6. The method of any one of claims 1 to 5, wherein the target map is based upon a brain image or scan of the subject.

7. The method of any one of claims 1 to 5, wherein the target map is based on common locations of protein deposits for the subject's disease/disorder.

8. The method of claim 6, wherein the image or scan is selected from CT, fMRI, fNIRS, MRI, PET, rs-fcMRI, and SPECT or a combination thereof.

9. The method of claim 8, wherein the image or scan comprises use of an imaging tracer.

10. The method of claim 9, wherein the imaging tracer identifies protein deposits.

11. The method of claim 9 or claim 10, wherein the imaging tracer identifies amyloid and/or Tau protein deposits.

12. The method of any one of claims 9 to 11, wherein the imaging tracer is selected from Florbetaben (Neuraceq®); Florbetapir (Amyvid®); Flutemetamol (Vizamyl®); Pittsburgh compound B (PIB), e.g., [¹¹C]PiB; [¹¹C]PBR28; and [¹⁸F]T807.

13. The method of any one of claims 8 to 12, wherein the image or scan is PET.

14. The method of any one of claims 6 to 13, wherein the image or scan is selected from EEG, ERPs, MEG, theta-burst rTMS, TMS/EEG, and TMS/MEPs, or a combination thereof.

15. The method of claim 14, wherein EEG and/or MEG is used to determine the stimulation waveform(s) and/or spatiotemporal stimulation pattern.

16. The method of any one of claims 7 to 15, wherein the target map is based on brain images or scans from a cohort of subjects.

17. The method of any one of claims 1 to 16, wherein the target map defines a desired spatiotemporal stimulation pattern for the subject.

18. The method of claim 17, wherein an electrode montage is selected to deliver to spatiotemporal stimulation pattern, optionally using a genetic algorithm.

19. The method of any one of claims 1 to 18, wherein the stimulation waveform is in the quasi-static regime of less than about 10,000 Hz.

20. The method of any one of claims 1 to 19, wherein the stimulation waveform is in the gamma band.

21. The method of any one of claims 1 to 19, wherein the stimulation comprises more than one distinct stimulation waveform.

22. The method of claim 21, wherein the more than one distinct stimulation waveform comprises at least one stimulation waveform in the gamma band.

23. The method of claim 22, wherein at least one stimulation waveform is outside the gamma band.

24. The method of claim 22, wherein each stimulation waveform is in the gamma band.

25. The method of any one of claims 21 to 24, wherein at least one stimulation waveform is a non-sinusoidal waveform.

26. The method of claim 25, wherein the non-sinusoidal waveform is in the gamma band.

27. The method of any one of claims 21 to 26, wherein the stimulation comprises random and/or varying frequencies.

28. The method of any one of claims 20 to 27, wherein the gamma band is between about 30 Hz and about 120 Hz.

29. The method of claim 28, wherein the gamma band is between about 40 Hz and about 50 Hz.

30. The method of any one of claims 1 to 29, wherein the current intensity is between about 0.1 mA and about 10 mA or between about 0.1 A/m² to about 100 A/m².

31. The method of any one of claims 1 to 30, wherein the duration is at least 1 seconds, at least 1 minute, or at least 1 or 2 hours, e.g., from about 5 minutes to about 1 hour.

32. The method of any one of claims 1 to 31, wherein the regimen comprises one session.

33. The method of any one of claims 1 to 31, wherein the regimen comprises more than one session, with the sessions being annual, bimonthly, monthly, semimonthly, biweekly, weekly, semiweekly, daily, or more than daily, and any number of periodic sessions therebetween.

34. The method of any one of claims 1 to 33, wherein the NIBS is provided via at least one electrode, TMS coil, or acoustic lens.

35. The method of any one of claims 1 to 34, wherein the NIBS is provided via an electrode montage, TMS coil montage, or acoustic lens montage comprising, respectively, more than one electrode, TMS coil, or acoustic lens.

36. The method of claim 35, wherein at least two electrodes, TMS coils, or acoustic lenses in the, respectively, electrode montage, TMS coil montage, or acoustic lens montage have different stimulation waveform and/or intensities.

37. The method of claim 35, wherein each electrode, TMS coil, or acoustic lens in the, respectively, electrode montage, TMS coil montage, or acoustic lens montage has the same stimulation waveform and/or current intensity.

38. The method of any one of claims 35 to 37, wherein the electrode montage, TMS coil montage, or acoustic lens montage, respectively, comprises up to 2, up to 4, up to 8, up to 16, up to 32, up to 64, up to 128, or up to 256 electrodes, TMS coils, or acoustic lenses.

39. The method of any one of claims 1 to 38, wherein the NIBS is provided via transcranial current stimulation (tCS), transcranial magnetic stimulation (TMS), or transcranial focused ultrasound (FUS).

40. The method of claim 39, wherein the tCS is selected from transcranial direct current stimulation (tDCS), transcranial alternating current stimulation (tACS), random noise current stimulation (tRNS), general field stimulation (gF-tCS), or a variant where each electrode is configured to stimulate with a unique, independent, and arbitrary waveform.

41. The method of claim 39 or claim 40, wherein the tCS is tACS.

42. The method of claim 40 or claim 41, wherein the tACS a multichannel tACS.

43. The method of claim 42, wherein at least two channels in the multichannel tACS have different stimulation waveforms and/or intensities.

44. The method of claim 42, wherein each channel has the same stimulation waveform and/or current intensity.

45. The method of any one of claims 1 to 44, wherein the stimulation is directed to at least one location outside of the auditory cortex and/or the visual cortex.

46. The method of any one of claims 1 to 45, wherein the stimulation's electric field is perpendicular or normal to the surface of the cortex of the brain of the subject.

47. The method of any one of claims 1 to 46, wherein the stimulation entrains gamma activity in the brain of the subject.

48. The method of any one of claims 1 to 47, wherein the subject has a disease associated with protein deposits.

49. The method of claim 48, wherein the disease associated with protein deposits is selected from Alzheimer's disease (AD), amyotrophic lateral sclerosis (ALS), dentatorubral-pallidoluysian atrophy (DRPLA), familial amyloid cardiomyopathy (FAC), familial amyloid polyneuropathy (FAP), Huntington disease (HID), mild cognitive impairments (MCI), Parkinson's disease (PD), prion diseases or transmissible spongiform encephalopathies (TSEs), senile systemic amyloidosis (SSA), spinal bulbar muscular atrophy (SBMA), spinocerebellar ataxia type 1 (SCA1), spinocerebellar ataxia type 3 (SCA3), spinocerebellar ataxia type 6 (SCA6), spinocerebellar ataxia type 7 (SCA7), and traumatic brain injury (TBI), or a combination thereof.

50. The method of claim 48 or claim 49, wherein the protein deposits are amyloid and/or Tau protein deposits, and the subject has Alzheimer's disease

51. The method of any one of claims 48 to 50, wherein the subject is undergoing or has undergone a pharmaceutical therapy for Alzheimer's disease.

52. The method of any one of claims 1 to 47, wherein the subject is at risk for developing or is asymptomatic for a disease associated with protein deposits.

53. A method for reducing protein deposits in the brain of a subject comprising:
creating a target map comprising actual location(s) of protein deposits in the subject's brain and densities thereof and/or likely location(s) of protein deposits in the subject's brain;
determining appropriate transcranial current stimulation (tCS) stimulation parameters to target the actual location(s) and/or likely location(s) of protein deposits; and
providing a non-invasive brain stimulation (NIBS) under the appropriate tCS stimulation parameters to target the actual location(s) and/or likely location(s) of protein deposits, thereby reducing the protein deposits.

54. A method comprising:
creating a target map comprising actual location(s) of protein deposits in a subject's brain and densities thereof and/or likely location(s) of protein deposits; and
determining appropriate non-invasive brain stimulation (NIBS) parameters to target the actual location(s) and/or likely location(s) of protein deposits; and
wherein targeting the actual location(s) of protein deposits with the appropriate NIBS stimulation parameters reduces the protein deposits.

55. A method for treating a symptom of a disease associated with protein deposits:
obtaining a target map, wherein the target map identifies actual location(s) of protein deposits in the brain of a subject and/or likely location(s) for protein deposits in the brain of the subject; and
providing a non-invasive brain stimulation (NIBS) to the actual location(s) and/or likely location(s) in a duration; stimulation waveform(s); spatiotemporal pattern; stimulation intensity; number and type of electrode, transcranial magnetic stimulation (TMS) coil, or acoustic lens; and/or regimen sufficient to reduce one or more protein deposit(s), thereby treating a symptom of the disease associated with protein deposits.

56. A method for preventing a symptom of a disease associated with protein deposits:
obtaining a target map, wherein the target map identifies likely location(s) for protein deposits in the brain of the subject and/or common location(s) for protein deposits in the brain of a subject with the disease associated with protein deposits; and
providing a non-invasive brain stimulation (NIBS) to the likely location(s) and/or common location(s) in a duration; stimulation waveform(s); spatiotemporal pattern; stimulation intensity; number and type of electrode, transcranial magnetic stimulation (TMS) coil, or acoustic lens; and/or regimen sufficient to reduce one or more protein deposit(s), thereby preventing a symptom of the disease associated with protein deposits.

57. A method for reducing one or more protein deposit(s) in the brain of a subject comprising providing a non-invasive brain stimulation (NIBS) directed to at least one location outside of the auditory cortex and/or the visual cortex in a duration; stimulation waveform(s); spatiotemporal pattern; stimulation intensity; number and type of electrode, transcranial magnetic stimulation (TMS) coil, or acoustic lens; and/or regimen sufficient to reduce one or more protein deposit(s).

58. A method for reducing amyloid and/or Tau protein deposits in the brain of a subject comprising:
obtaining a target map comprising actual location(s) of amyloid and/or Tau protein deposits in the subject's brain and densities thereof and/or likely location(s) of amyloid and/or Tau protein deposits in the subject's brain; and
providing a non-invasive brain stimulation (NIBS) to the actual location(s) and/or likely location(s) in a duration; stimulation waveform(s); spatiotemporal pattern; stimulation intensity; number and type of electrode, transcranial magnetic stimulation (TMS) coil, or acoustic lens; and/or regimen sufficient to reduce the amyloid and/or Tau protein deposits.

59. A method for reducing amyloid and/or Tau protein deposits in the brain of a subject comprising:
creating a target map comprising actual location(s) of amyloid and/or Tau protein deposits in the subject's brain and densities thereof and/or likely location(s) of amyloid and/or Tau protein deposits in the subject's brain;
determining appropriate transcranial alternating current stimulation (tACS) stimulation parameters to target the actual location(s) and/or likely location(s) of amyloid and/or Tau protein deposits; and
providing a non-invasive brain stimulation (NIBS) under the appropriate tACS stimulation parameters to target the actual location(s) and/or likely location(s) of amyloid and/or Tau protein deposits, thereby reducing the amyloid and/or Tau protein deposits.

60. A method comprising:
creating a target map comprising actual location(s) of amyloid and/or Tau protein deposits in a subject's brain and densities thereof and/or likely location(s) of amyloid and/or Tau protein deposits; and
determining appropriate non-invasive brain stimulation (NIBS) parameters to target the actual location(s) and/or likely location(s) of amyloid and/or Tau protein deposits; and
wherein targeting the actual location(s) of amyloid and/or Tau protein deposits with the appropriate NIBS stimulation parameters would reduce the amyloid and/or Tau protein deposits.

61. A method for treating a symptom of Alzheimer's disease associated with amyloid and/or Tau protein deposits:
obtaining a target map, wherein the target map identifies actual location(s) amyloid and/or Tau protein deposits in the brain of a subject and/or likely location(s) for amyloid and/or Tau protein deposits in the brain of the subject; and
providing a non-invasive brain stimulation (NIBS) to the actual location(s) and/or likely location(s) in a duration; stimulation waveform(s); spatiotemporal pattern; stimulation intensity; number and type of electrode, transcranial magnetic stimulation (TMS) coil, or acoustic lens; and/or regimen sufficient to reduce the amyloid and/or Tau protein deposits, thereby treating a symptom of Alzheimer's disease.

62. A method for preventing a symptom of Alzheimer's disease associated with amyloid and/or Tau protein deposits:
obtaining a target map, wherein the target map identifies likely location(s) for amyloid and/or Tau protein deposits in the brain of the subject and/or common location(s) for amyloid and/or Tau protein deposits in the brain of a subject with Alzheimer's disease; and
providing a non-invasive brain stimulation (NIBS) to the likely location(s) and/or common location(s) in a duration; stimulation waveform(s); spatiotemporal pattern; stimulation intensity; number and type of electrode, transcranial magnetic stimulation (TMS) coil, or acoustic lens; and/or regimen sufficient to reduce an amyloid and/or Tau protein deposit, thereby preventing a symptom of Alzheimer's disease.

63. A method for reducing protein deposits in the brain of a subject comprising:
obtaining a target map comprising the subject's brain developed from positron emission tomography (PET) data and from MRI data collected from the subject; and
providing a non-invasive brain stimulation (NIBS) by multichannel transcranial alternating current stimulation (tACS), with electrode placement and stimulation parameters chosen to engage the target map using a standard head model, wherein one or more stimulation frequencies are in the gamma band.

64. A method for preventing or treating a symptom of Alzheimer's disease associated with amyloid and/or Tau protein deposits in the brain of a subject comprising providing a non-invasive brain stimulation (NIBS) directed to at least one location outside of the auditory cortex and/or the visual cortex in a duration; stimulation waveform(s); spatiotemporal pattern; stimulation intensity; number and type of electrode, transcranial magnetic stimulation (TMS) coil, or acoustic lens; and/or regimen sufficient to reduce one or more protein deposit(s).

65. A method for preventing or treating a symptom of a disease involving cognitive deficits associated with impaired activity in the gamma range in cortical circuits,
the method comprising a step of providing a non-invasive brain stimulation (NIBS) directed to at least one location having cortical circuits with impaired activity in the gamma range in a duration; stimulation waveform(s); spatiotemporal pattern; stimulation intensity; number and type of electrode, transcranial magnetic stimulation (TMS) coil, or acoustic lens; and/or regimen sufficient to reduce one or more protein deposit(s).

66. The method of claim 65, wherein the impaired gamma activity is in at least interneurons in the cortical circuit.

67. The method of claim 65 or claim 66, wherein the disease is characterized by deficits in cognitive control.

68. The method of any one of claims 65 to 68, wherein the disease is schizophrenia.

69. A method for preventing or treating a symptom of a disorder involving deficits in neuronal circuitry related to gamma oscillations,
the method comprising a step of providing a non-invasive brain stimulation (NIBS) directed to at least one location having cortical circuits with impaired activity in the gamma range in a duration; stimulation waveform(s); spatiotemporal pattern; stimulation intensity; number and type of electrode, transcranial magnetic stimulation (TMS) coil, or acoustic lens; and/or regimen sufficient to reduce one or more protein deposit(s).

70. The method of claim 69, wherein the deficits in gamma oscillations occurs in at least interneurons in the neuronal circuit.

71. The method of claim 69 or claim 70, wherein the disorder is autism.
